(19) United States
(12) Patent Application Publication
(10) Pub. No.: US 2025/0032283 A1
(43) Pub. Date: Jan. 30, 2025

(54) DEVICES AND METHODS FOR STENT GRAFT EXTRACTION

(71) Applicant: Hjarta Care, LLC, Waukesha, WI (US)

(72) Inventor: BRIAN THORSON, Mequon, WI (US)

(73) Assignee: Hjarta Care, LLC, Waukesha, WI (US)

(21) Appl. No.: 18/783,773

(22) Filed: Jul. 25, 2024

Related U.S. Application Data
(60) Provisional application No. 63/529,034, filed on Jul. 26, 2023.

(51) Int. Cl. A61F 2/95

(52) U.S. Cl. CPC A61F 2/9517; A61F 2002/9528

(57) ABSTRACT

A device for extracting an endovascular implant from a vessel includes a cylindrical body, a trough, and a handle. The cylindrical body includes a distal opening, an opposing opening opposite the distal opening, and a side wall surrounding a hollow bore of the cylindrical body. The trough extends from the cylindrical body proximate the opposing opening and includes a first side wall and a second side wall. The handle extends from the trough. A thickness of the sidewall at the distal opening tapers toward the opposing opening such that the hollow bore proximate the distal opening is narrower than the hollow bore proximate the opposing opening.

### CROSS-REFERENCE TO RELATED APPLICATION

This U.S. patent application claims the benefit of and priority to U.S. Provisional Application No. 63/529,034, filed Jul. 26, 2023, the entire disclosure of which is hereby incorporated by reference herein.

### BACKGROUND

The present disclosure relates generally to the field of endovascular stent graft and other implant extraction, and more particularly to devices and methods for atraumatic extraction of stent grafts and other circulatory system implants.

An endovascular stent graft may be used for a variety of conditions involving the blood vessels, but most commonly to reinforce a weak spot in an artery called an aneurysm. Over time, blood pressure and other factors can cause this weak area to bulge and eventually enlarge and rupture. A stent graft is implanted to tightly seal with the artery above and below the aneurysm. The graft is stronger than the weakened artery and allows blood to pass through it without pushing on the bulge.

Occasionally, extraction of the stent graft is necessary due to infection or failure of the original implant to perform as intended. Because the stent graft typically includes prongs which engage with the vessel wall, extraction of the device can cause significant damage to the tissue to which it is engaged. Stent graft explant is known to be associated with high morbidity, caused by a confluence of factors. Damage caused to the vessel wall during the extraction process is one factor that contributes to the high morbidity.

Accordingly, there is a need for a device that promotes atraumatic removal of a stent graft (or other graft) from a vessel and/or of other structures that may have been implanted in a circulatory system of a patient, such as valves (e.g., aortic and pulmonary valves).

### SUMMARY

One embodiment is directed towards a device for extracting an implant from a vessel. The device includes a cylindrical body, a trough, and a handle. The cylindrical body includes a distal opening, an opposing opening opposite the distal opening, and a side wall surrounding a hollow bore of the cylindrical body. The trough extends from the cylindrical body proximate the opposing opening and includes a first side wall and a second side wall. The handle extends from the trough. A thickness of the sidewall at the distal opening tapers toward the opposing opening such that the hollow bore proximate the distal opening is narrower than the hollow bore proximate the opposing opening such that a diameter of the opposing opening is larger than a diameter of the distal opening.

Another embodiment is directed toward a method for extracting an implant from a vessel. The method includes inserting an extraction device into the vessel. The extraction device includes a cylindrical body with a distal opening, an opposing opening opposite the distal opening, and a sidewall surrounding a hollow bore. The extraction device also includes a trough extending from the cylindrical body proximate the opposing opening having a first side wall and a second side wall. The extraction device also includes a handle extending from the trough. A thickness of the sidewall proximate the distal opening tapers toward the opposing opening such that the hollow bore proximate the distal opening is narrower than the hollow bore proximate the opposing opening. The method also includes sliding the extraction device over the implant until the extraction device causes a prong of the implant to release from the vessel. The method also includes removing at least one of the implant or the extraction device from the vessel while the sidewall of the extraction device is located at least partially between the implant and the vessel. In some embodiments, the implant is an arterial stent graft. In other embodiments, the implant is a replacement valve.

Yet another embodiment is directed toward a device for extracting an endovascular implant from a vessel. The device includes a cylindrical body and a trough. The cylindrical body includes a distal opening, an opposing opening opposite the distal opening, and a side wall surrounding a hollow bore of the cylindrical body. The trough extends from the cylindrical body proximate the opposing opening and includes a first side wall and a second side wall. A thickness of the sidewall at the distal opening tapers toward the opposing opening such that the hollow bore proximate the distal opening is narrower than the hollow bore proximate the opposing opening. The first side wall and the second side wall taper away from the cylindrical body such that a depth of the trough is greater proximate the cylindrical body than away from the cylindrical body.

### BRIEF DESCRIPTION OF THE DRAWINGS

A clear conception of the advantages and features constituting the present disclosure, and of the construction and operation of typical mechanisms provided with the present disclosure, will become more readily apparent by referring to the exemplary, and therefore non-limiting, embodiments illustrated in the drawings accompanying and forming a part of this specification, wherein like reference numerals designate the same elements in the several views, and in which:

FIG. 1A is a perspective view of an endovascular stent graft for use in an abdominal aorta, according to an exemplary embodiment.

FIG. 1B is a partial view of the endovascular stent graft of FIG. 1 implanted into the abdominal aorta, according to an exemplary embodiment.

FIG. 2 is a side view of an endovascular extraction device, according to a first exemplary embodiment.

FIG. 3 is a top, front, perspective view of the endovascular extraction device of FIG. 2, according to an exemplary embodiment.

FIG. 4 is a side view of the endovascular extraction device of FIG. 2, according to an exemplary embodiment.

FIG. 5 is a top view of the endovascular extraction device of FIG. 2, according to an exemplary embodiment.

FIG. 6 is a top view of an endovascular stent graft for use in an abdominal aorta, according to an exemplary embodiment.

FIG. 7A is a side, section view of the endovascular extraction device of FIG. 2 taken about plane A-A, according to an exemplary embodiment.

FIG. 7B is a detailed cross-section view of the sidewall of the ring of the endovascular extraction device as shown in FIG. 7A taken at Detail B, according to an exemplary embodiment.

FIG. 8 is a side view of an endovascular extraction device, according to an exemplary embodiment.

FIG. 9 is a top, front, perspective view of the endovascular extraction device of FIG. 8, according to an exemplary embodiment.

FIG. 10 is a side view of the endovascular extraction device of FIG. 8, according to an exemplary embodiment.

FIG. 11 is a side view of an endovascular extraction device having a concave handle, according to an exemplary embodiment.

FIG. 12 is a top view of the endovascular extraction device of FIG. 8, according to an exemplary embodiment.

FIG. 13A is a side, section view of the endovascular extraction device of FIG. 8 taken about plane C-C, according to an exemplary embodiment.

FIG. 13B is a detailed cross-section view of the sidewall of the ring of the endovascular extraction device as shown in FIG. 13A taken at Detail D, according to an exemplary embodiment.

FIGS. 14A-14D depict the endovascular stent graft extraction device of FIG. 2, FIG. 8, or FIG. 11 being used to extract an endovascular stent graft, according to an exemplary embodiment.

The foregoing and other features of the present disclosure will become apparent from the following description and appended claims, taken in conjunction with the accompanying drawings. Understanding that these drawings depict only several embodiments in accordance with the disclosure and are therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

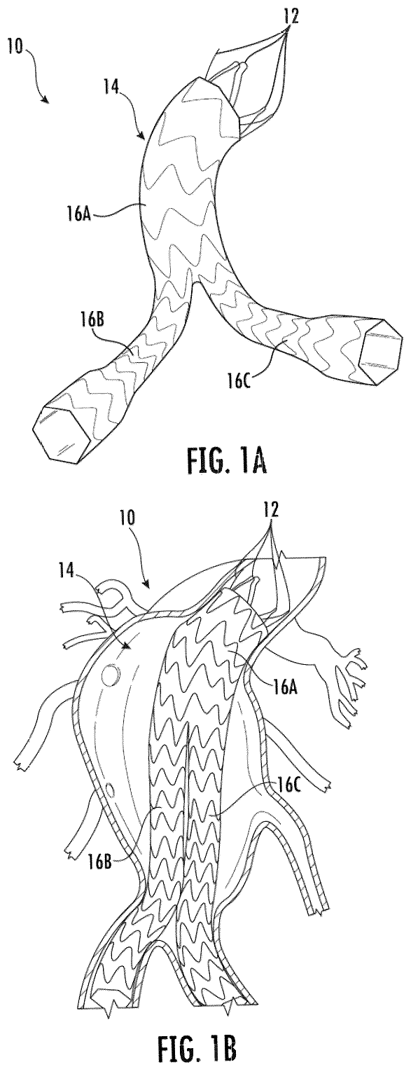

Referring generally to the figures, described herein is an endovascular extraction device. According to an embodiment, the endovascular extraction device includes a distal ring, a trough, and a handle portion. At a distal end of the distal ring is an opening. Adjacent to the distal ring is the trough, which leads to the handle portion. The trough follows an upward curve as it extends to the handle portion. The terminal end of the handle also includes a curve. The embodiments of an endovascular extraction device described herein are configured for removal of an endovascular stent graft as shown and described with reference to FIGS. 1A-1B, and/or removal of an endovascular implant (e.g., a replacement valve).

Referring to FIGS. 1A-1B, a stent graft 10 (e.g., endovascular implant, etc.) is shown. The stent graft 10 is configured to be implanted within one or more various arteries of a person, for example a patient with an aneurysm, to reinforce the walls of the artery. In some embodiments, the stent graft 10 is specifically configured to be used in the abdominal aorta and the iliac arteries branching off of the abdominal aorta (FIG. 1B). The stent graft 10 includes prongs 12 and a frame 14 having an aortic portion 16A and one or more artery portions (e.g., branches) 16B and 16C. Each portion may further include a respective central axis (not shown) along which the frame extends. The frame 14 can be made of a variety of materials configured to be implanted within arteries and provides the support to reinforce the walls of the arteries from bursting. Additionally, to secure the stent graft 10, the frame 14 may be configured to collapse and expand along a respective central axis (e.g., the length of the frame 14 can change) but to be biased in such a way that it acts rigid or solid radial to the central axis (e.g., the diameter of the portions of the frame 14 does not change). In this way, the frame 14 supports the walls of the arteries (e.g., the abdominal aorta and the iliac arteries branching off of the abdominal aorta) but is able to be expanded and contracted for implantation. Similarly, the frame 14 includes the aortic portion 16A (a relatively wide portion) and the one or more artery portions 16B and 16C (relatively narrower portions). In other embodiments, the frame 14 may include other portions (e.g., more branches, fewer branches, no aortic portion, etc.) depending on where the stent graft 10 is to be used. The frame 14 may have varied diameters depending on the size of vessel in which the stent graft 10 is placed.

The prongs 12 are coupled to the aortic portion 16A and are configured to selectively move between a deployed position in which they press against the walls of the artery and prevent movement of the stent graft 10 and a non-deployed position in which they do not press against the walls of the artery and do not prevent movement of the stent graft 10. In some embodiments, the prongs are coupled to other portions of the frame 14. In the deployed position (FIGS. 1A and 1B), the prongs 12 extend at least partially radially outward from the central axis of the aortic portion 16A and press against the walls of the aorta (or artery) to prevent the stent graft 10 from moving. By doing so, the prongs 12 may dig in or provide a friction force that keeps the stent graft 10 in place. While being implanted, a special device may be required to move the prongs 12 into the deployed position. In the non-deployed position (not shown), the prongs 12 do not extend radially outward or contact the walls of the aorta. In the non-deployed position, the stent graft 10 is able to be moved around within the arteries to be correctly positioned to cover the aneurysm. Once in place, the prongs 12 may be selectively moved to the deployed position and “implanted” within the aorta. At this point, the stent graft 10 can be left in the aorta for long periods of time (e.g., permanently, multiple years, etc.) without moving to prevent the walls of the aorta (or arteries) from rupture.

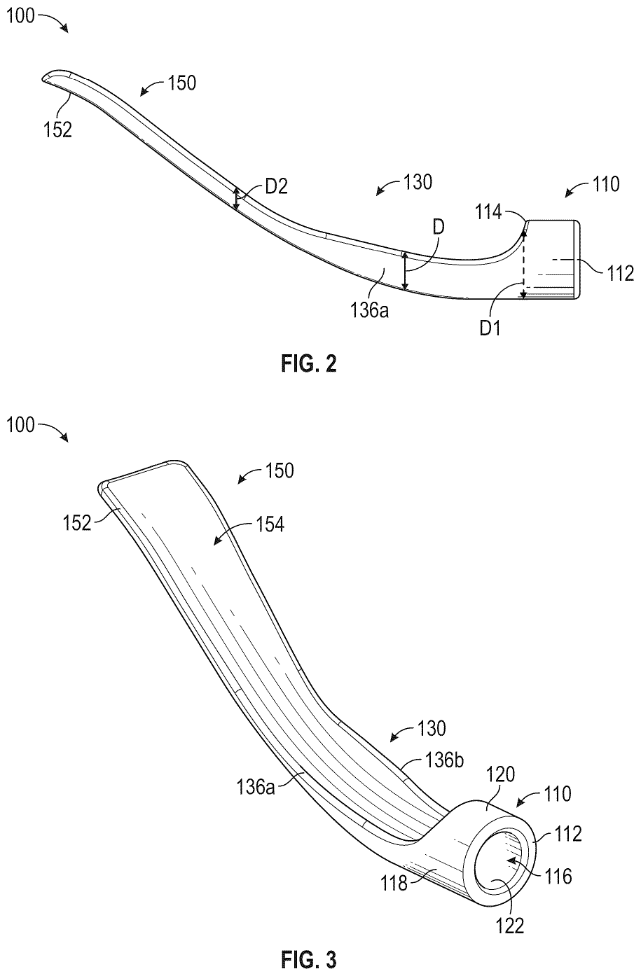

Referring now to FIGS. 2-3 and 8-9, a extraction device 100 (e.g., endovascular stent graft extraction devices, etc.) is shown, according to exemplary embodiments. While the stent graft 10 is configured to be left in the aorta (or arteries) for long periods of time, various complications may develop (e.g., infection, the stent graft 10 not deploying correctly, swelling of the arteries, plaque buildup, failure, etc.) which require the removal of the stent graft 10. The extraction device 100 is therefore configured to be inserted within the arteries and to be used to extract the stent graft 10 atraumatically (e.g., with little to no damage to the arteries themselves).

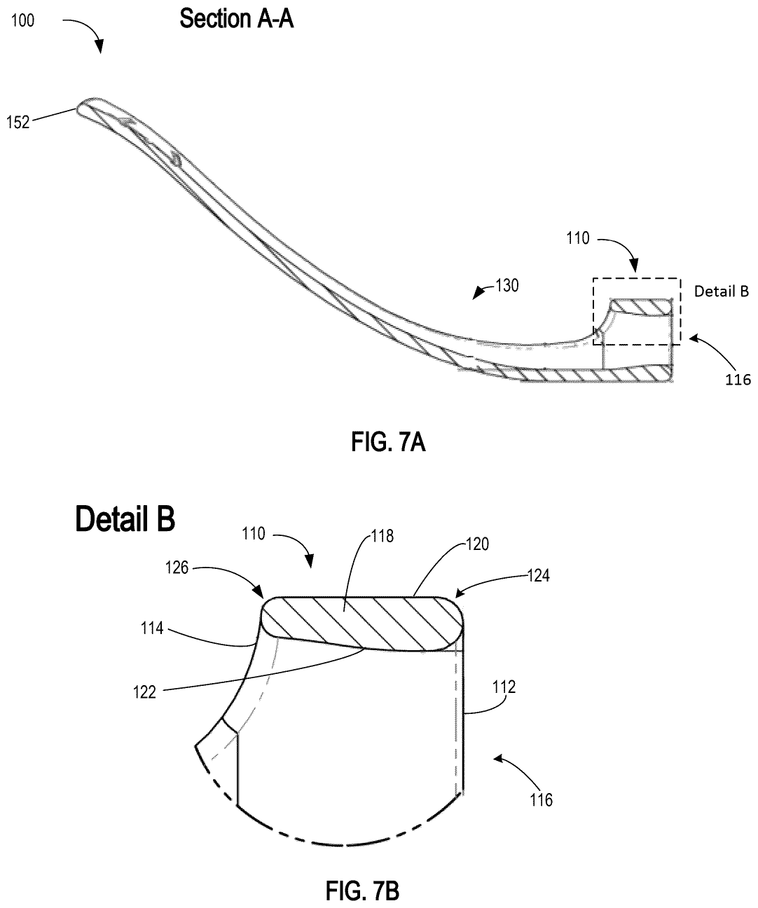

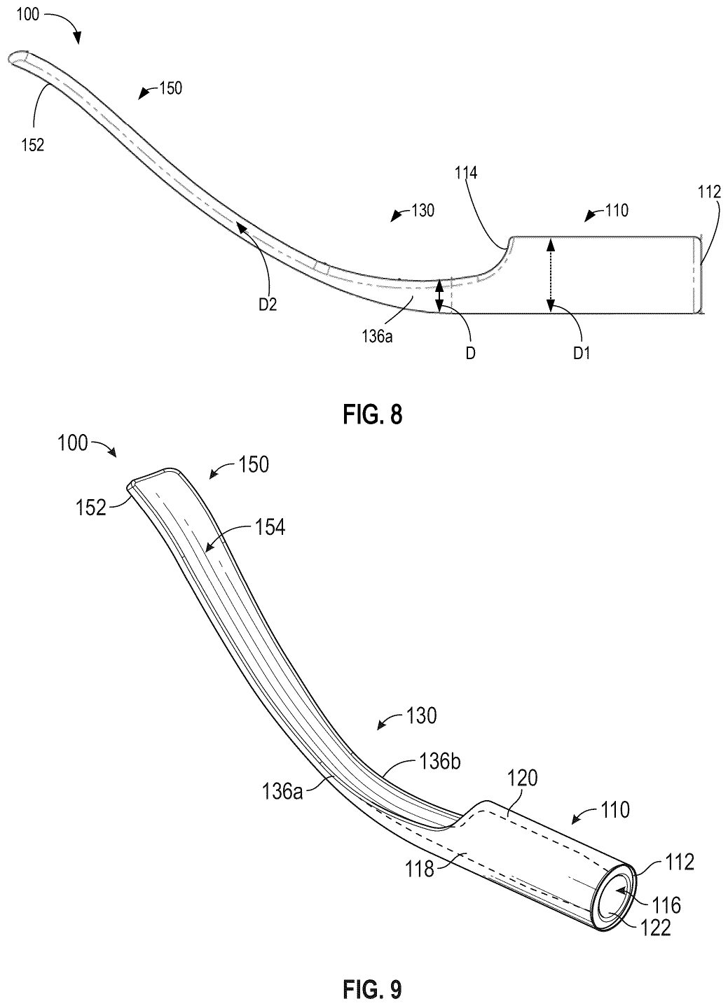

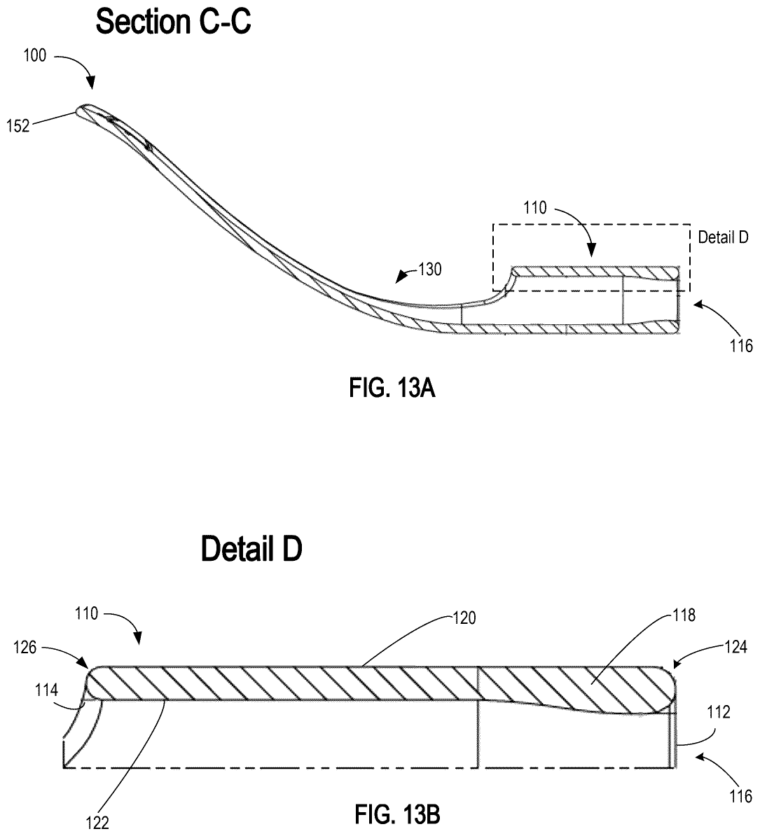

According to an embodiment, the extraction device 100 includes a distal ring 110, a trough 130, and a handle 150. The distal ring 110 has a cylindrical body, a distal opening 112 providing access to a hollow bore 116 of the distal ring 110, an opposing opening 114 opposite the distal opening 112 and also providing access to the hollow bore 116 of the distal ring 110, and a sidewall 118 surrounding the hollow bore 116. According to an embodiment, the distal ring 110 and the distal opening 112 comprise a circular cross-sectional shape. According to an embodiment, the distal ring 110 includes an outer diameter that is sized with respect to the aorta or artery, such that it is at least slightly smaller than the diameter of the walls of the aorta or artery. Various sizes may be provided to accommodate different patients (e.g., having a specific diameter of a distal ring 110 for a specific patient, etc.). Generally, the outer diameter (e.g., measured at an outer surface 120 of sidewall 118) of the distal ring 110 is approximately 19-33 mm, or approximately 22-29 mm, or approximately 25-26 mm. The inner diameter (e.g., measured at a inner surface 122 of sidewall 118) of the distal ring 110 is approximately 11-25 mm, or approximately 14-22 mm, or approximately 15-16 mm, and may vary along the longitudinal (e.g., axial) dimension of the cylindrical body forming the distal ring 110, as discussed in further detail below with reference to FIGS. 7A-7B and 13A-13B. According to the exemplary embodiment shown in FIGS. 2-3, the length of the cylindrical body forming the distal ring 110 (e.g., in the longitudinal/axial direction) is approximately 14-18 mm, or approximately 15-17 mm, or approximately 16 mm. According to the exemplary embodiment shown in FIGS. 8-9, the length of the cylindrical body forming the distal ring 110 (e.g., in the longitudinal/axial direction) is approximately 48-52 mm, or approximately 49-51 mm, or approximately 50 mm. In other embodiments, the length of the cylindrical body forming the distal ring 110 may be other lengths.

In exemplary embodiments, the extraction device 100 may be used to extract various types of grafts and implants. For example, the extraction device 100 may be inserted into an aorta of a patient to remove a replacement valve placed by a transcatheter aortic valve replacement procedure (TAVR). TAVR procedures involve inserting a catheter holding a replacement valve into the aorta of a patient. Once the catheter reaches the aortic valve, the replacement valve is deployed. In some examples, the replacement valve is inflated (e.g., by a balloon catheter) to expand the walls of the replacement valve. In other examples, the replacement valve is self-expanding. While replacement valves are configured to be left in the aortic valve for long periods of time, various complications may develop (e.g., infection, breakdown of the organic replacement valve tissues, swelling of the aorta, plaque buildup, failure, etc.) which require the removal of the valve replacement. The extraction device 100 is therefore configured to be inserted into the aorta to extract replacement valves atraumatically (e.g., with little to no damage to the aorta or other surrounding tissues including heart tissues).

Once inserted into the aorta, the extraction device 100 may be slid (e.g., pushed, moved, etc.) along the wall of the aorta until it reaches the edges of the replacement valve. At this point, the distal ring 110 moves between the wall of the aorta, the wall of the patient's aortic valve, and the external wall of the replacement valve. The replacement valve moves into the hollow bore 116, and eventually into the trough 130 as the extraction device 100 continues to move within the aorta until it reaches the left ventricle. At this point, the replacement valve is removed from the walls of the aorta and aortic valve, and the user of the extraction device may remove the extraction device 100 through the aorta. Although this example relates to removal of a replacement aortic valve, the extraction device may be similarly used to remove other replacement valves (e.g., mitral, tricuspid, and pulmonary replacement valves) or implants (e.g., iliac vein stents, implantable venous access devices, venous system implants, etc.).

The length of the distal ring 110 may vary to accommodate various lengths of the type of device being explanted (e.g., replacement valves, iliac graft, venous system implant, etc.). For example, the length of the distal ring 110 may be 16 mm for removal of replacement valves, 15 mm for the removal of iliac stent grafts, and 14 mm for the removal of venous system implants. Additionally or alternatively, the outer diameter of the distal ring may vary based on the type of device being explanted. The shape of the handle 150 may vary (e.g., convex curvature, concave curvature, straight, etc.) to complement the curvature of the anatomical geometry surrounding an implant. For example, the handle 150 may define a convex curvature for removal of replacement valves. As another example, the handle 150 may define a concave curvature for extraction of arterial stent grafts. The radius of curvature and angle defined by the handle may also be adjusted to complement various extraction applications.

Referring still to FIGS. 2-3 and 8-9, according to an embodiment, a trough 130 extends between the distal ring 110 and the handle 150. The trough has a varying depth D along its length, as a result of a curvature in the extraction device 100 at the trough 130, discussed below. The trough 130 further includes a plurality of side walls 136. For example, the trough 130 may include a first side wall 136a and a second side wall 136b. According to this embodiment, the second side wall 136b is positioned opposite of the first side wall 136a. At the location where the trough 130 extends from the distal ring 110, the trough has a depth D1 greater than a radius of the distal ring 110. In other words, the side walls 136a, 136b of the trough 130 extend up so far as to form greater than a half circle. The depth of the trough 130 decreases as the trough 130 extends towards the handle 150. For example, at a section proximate to the connecting location to the distal ring 110, the side walls 136a, 136b of the trough 130 extend up so far as to form approximately equal to a half circle. The trough 130 further extends to its minimum depth D2 at a location where the trough 130 transitions to the handle 150. The width of the trough 130 is discussed in further detail below with reference to FIGS. 5-6 and 11.

Referring still to FIGS. 2-3 and 8-9, according to an embodiment, the handle 150 extends from the trough 130 to a terminal end 152. The handle 150 includes a shallow valley 154. The depth of the shallow valley 154 is substantially constant along the length of the handle 150. In an embodiment, the depth of the shallow valley 154 is equal to the minimum depth D2 of the trough 130. The shallow valley 154 provides an ergonomic grasping design, for example, allowing a user's thumb to fit comfortably in the shallow valley 154 for grasping and manipulating the extraction device 100. As discussed in greater detail below, the terminal end 152 of the handle 150 includes a slight curvature. The width of the handle 150 is discussed in further detail below with reference to FIGS. 5-6 and 11.

The extraction device 100 may be made of a material that is rigid. The extraction device 100 may be made of a material that is suitable for sterilization (e.g., cleaning, purifying, etc.) such as surgical/medical grade steel, stainless steel, and surgical/medical grade plastic (e.g., polyethylene, polypropylene, etc.). In some embodiments, the extraction device 100 is made of medical grade resin. In some embodiments, the device is disposable (e.g, may be thrown away after a single use, etc.).

In some embodiments, the extraction device 100 is manufactured using 3D printing methods (e.g., Stereolithografty (SLA), Selective Laser Sintering (SLS), Fused Deposition Modeling (FDM), Direct Metal Laser Sintering (DMLS), or other conventional 3D printing methods). Beneficially, this provides the ability for one or more dimensions of the extraction device 100 to be easily changed or updated based on the specific aorta or arteries within which the device is working, or based on the specific stent graft 10 that is being extracted. While the aorta and arteries are typically a common diameter and length for a certain age and size of a patient, they may vary depending on genetics, prior surgeries, and other environmental factors. As a result, the extraction device 100 may be manufactured using 3D printing methods so the various dimensions may be easily and quickly updated, such as right on site at a surgical center or hospital. This is further accommodated by manufacturing the extraction device 100 as a disposable device (e.g., using medical grade resin), which requires far less finishing/polishing time and effort, as compared with a reusable version of the extraction device 100 made of, for example, stainless steel. Furthermore, a disposable version makes possible the availability of multiple diameter devices “off the shelf” and allows for custom made modifications to meet specific physician requests based on patient anatomy.

In use (e.g., to extract the stent graft 10, to extract a replacement valve, to extract a venous implant), according to an embodiment, the extraction device 100 is inserted into the aorta or the artery such that the distal ring 110 is located within the walls of the aorta or artery and controlled by a user gripping and manipulating the device from the handle 150. Once inserted into the aorta or artery, the extraction device 100 may be slid (e.g., pushed, moved, etc.) along the wall of the aorta until it comes to the frame 14 of the stent graft 10. At this point, as will be discussed further herein, the distal ring 110 comes between the wall of the aorta and the frame 14 such that the frame 14 moves into the hollow bore 116, and eventually into the trough 130 as the extraction device 100 continues to move within the artery. Once the distal ring 110 reaches the prongs 12, the extraction device 100 is configured to move the prongs 12 from the deployed position to the non-deployed position. At this point, the user of the extraction device 100 is able to extract the stent graft 10.

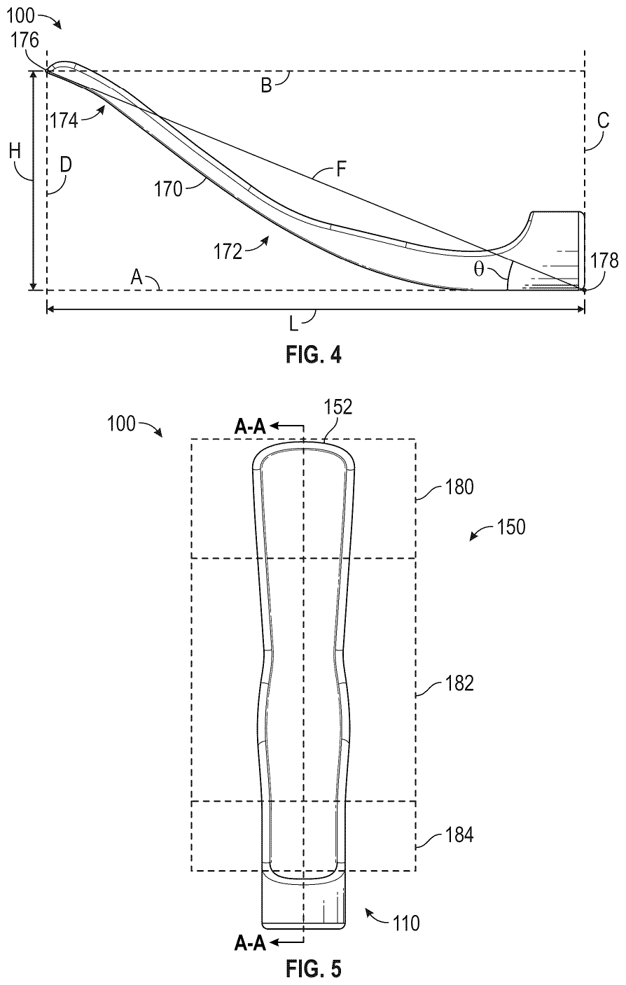

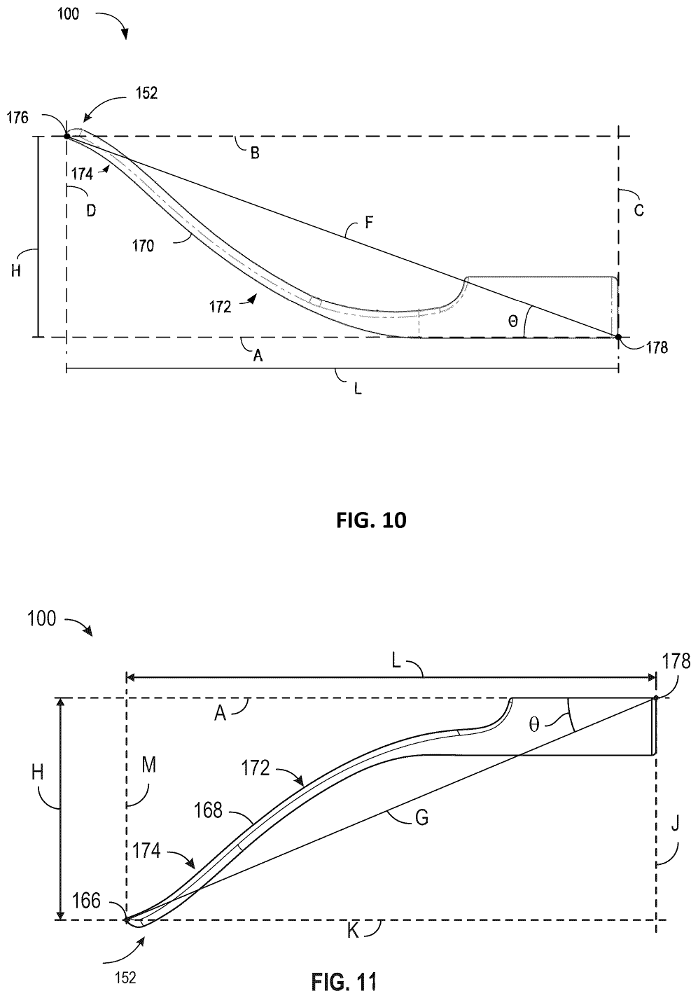

Referring now to FIGS. 4 and 10, in regards to further dimensions of an embodiment of the extraction device 100, the extraction device 100 includes a height (H) between a horizontal axis (A) aligned with a bottom surface 170 of the distal ring 110 and a horizontal line (B) aligned with an end point 176 of the handle 150. In some embodiments, the height H is approximately 60-80 mm, or approximately 65-70 mm, or approximately 68-70 mm. The extraction device 100 also includes a length L between a vertical axis (C) aligned with the end surface of the distal ring 110 and a vertical line (D) aligned with the end point of the handle 150. According to the exemplary embodiment shown in FIG. 4, the length L is approximately 130-160 mm, or approximately 140-150 mm, or approximately 144-146 mm. According to the exemplary embodiment shown in FIG. 10, the length L is approximately 165-195 mm, or approximately 175-185 mm, or approximately 179-181 mm. In other embodiments, the length L may be other lengths.

Referring still to FIGS. 4 and 10, the extraction device 100 has a bottom surface 170 which follows a first curvature 172 at the trough 130 and a second curvature 174 at the terminal end 152 of the handle 150. According to an embodiment, the first curvature 172 is a concave curvature relative to the trough 130 and the second curvature 174 is a convex curvature relative to the trough 130. The first curvature 172 of the handle 150 extends towards line (B), whereas the second curvature at the terminal end 152 of the handle 150 curves towards the horizontal, defined by axis (A) and line (B). The first curvature 172 has a radius of curvature of approximately 70 mm to 80 mm, or approximately 74 mm to 76 mm, or approximately 75 mm. As discussed above, the first curvature 172 in the bottom surface 170 of the extraction device 100 contributes to the varying depth of the trough 130, as the bottom surface 170 of the extraction device 100 at the trough raises above the axis (A) aligned with the bottom surface 170 of the extraction device 100 at the distal ring 110. The second curvature 174 is a slighter curve, having a smaller radius of curvature than the first curvature 172. As a result of the first curvature 172 and the second curvature 174, the extraction device 100 takes on a slide shape. The extraction device 100 also includes an angle Θ between the horizontal axis (A) and a line (F), connecting a point 178 on the bottom surface 170 at the distal vertical surface (C) of the distal ring 110 and the end point 176 of the handle 150. According to the exemplary embodiment shown in FIG. 4, the angle Q, is approximately 20-30 degrees, or approximately 24-26 degrees, or approximately 25 degrees. According to the exemplary embodiment shown in FIG. 10, the angle Θ is approximately 15-25 degrees, or approximately 19-21 degrees, or approximately 20 degrees. In some embodiments, the handle 150 is straight rather than defining a first or second curvature.

Referring now to FIG. 11, a side view of a extraction device 100 having a handle 150 which follows a first convex curvature is shown, i.e., with a handle 150 curving away from an opening in the distal ring 110 so as to have a substantially convex curvature from the perspective shown in FIG. 11. In some embodiments, the handle follows a second curvature at the terminal end 152. As shown, the first curve 172 of the handle 150 curves downwards, towards line (K) and away from line A (away from an opening in the distal ring 110), other than at a second curvature 174 at the terminal end 152 of the handle 150. In this example, the second curvature 174 curves towards (into parallel alignment with) a horizontal direction as shown in FIG. 11 (towards being parallel with axis (A) and line (K). The first curvature 172 has a radius of curvature of approximately 70 mm to 80 mm, or approximately 74 mm to 76 mm, or approximately 75 mm. Conversely to the convex curvature configuration, the first curvature 172 in the top surface 168 of the extraction device 100 does not contribute to the varying depth of the trough 130. Instead, the trough may be substantially horizontal. In some embodiments, the trough 130 defines a third curvature that is convex relative to the distal ring 110. The second curvature 174 is a slighter curve, having a smaller radius of curvature than the first curvature 172. As a result of the first curvature 172 and the second curvature 174, the handle 150 takes on a slide shape.

The extraction device 100 is also characterized by an angle Θ between the horizontal axis (A) and a line (G), connecting a point 178 on the bottom surface 170 at the distal vertical surface (J) of the distal ring 110 and the end point 166 of the handle 150. The angle Θ is approximately 20-30 degrees, or approximately 24-26 degrees, or approximately 25 degrees, according to some embodiments. The angle Θ may be approximately 15-25 degrees, or approximately 19-21 degrees, or approximately 20 degrees. In this example, the handle 150 takes a convex shape with a terminal end 166 that falls below the trough 130. Such curvature of the handle 150 in the example of FIG. 11 provides different (e.g., increased) line of sight to distal ring 110 as compared to other examples herein (e.g., FIG. 10), for example as may be suitable for different explant procedures associated with different anatomic geometries (e.g., for valve removals such as aortic valve implant removals). By forming the handle 150 in a convex shape, a user may have more visibility in some explant procedures; for example, the example of FIG. 11 may be used in removal of aortic valve implant (e.g., for correction of a transcatheter aortic valve replacement (TAVR)).

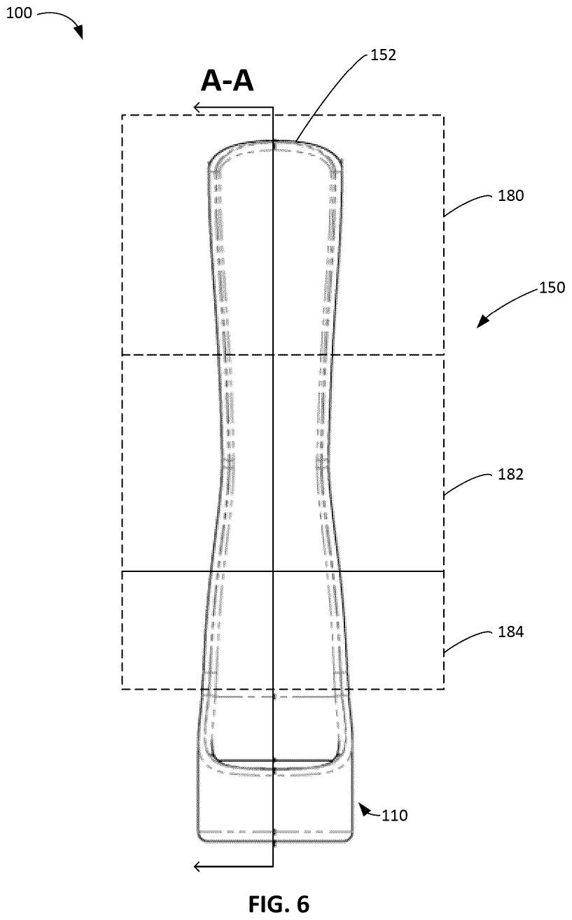

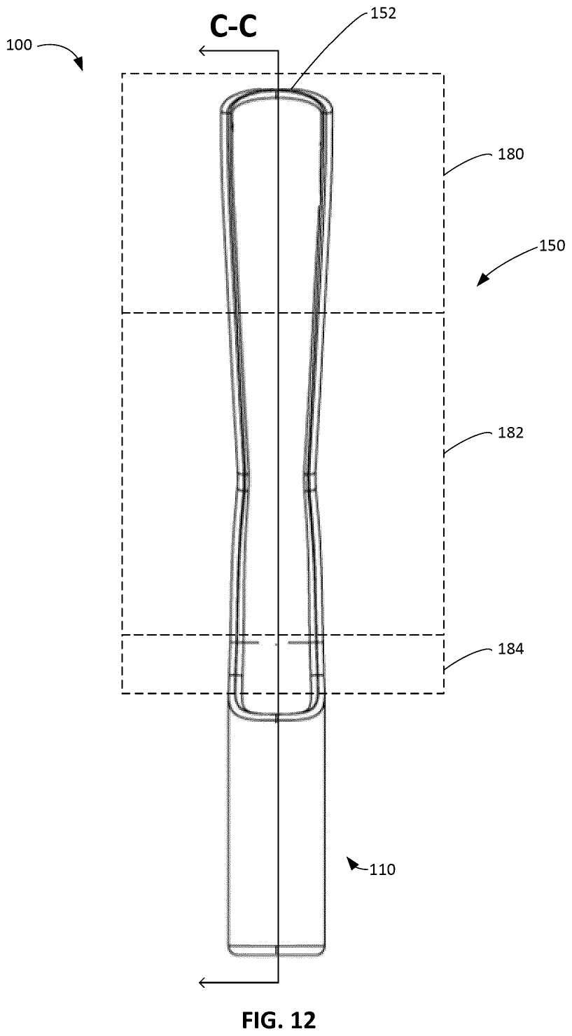

Referring now to FIGS. 5-6 and 12 which show a top view of the extraction device 100. As shown, the width of the extraction device 100 varies along its length, having somewhat of an hourglass figure. In particular, there are slight inward and outward bows in the side walls 136a, 136b of the trough 130. For example, the shallow valley 154 further includes a terminal portion 180, a central portion 182, and a distal portion 184. The terminal portion 180 is positioned near the terminal end 152. The distal portion 184 is positioned near the distal ring 110. The central portion 182 is positioned in between the terminal portion and the distal portion 184. According to the exemplary embodiment shown in FIG. 5, a diameter of the central portion 182 of the trough 130 proximate the distal portion 184 is slightly larger than the diameter of the distal ring 110, a diameter of the central portion 182 of the trough 130 proximate the terminal portion 180 is slightly smaller than the diameter of the distal ring 110, and a diameter of the terminal portion 180 of the trough 130 is slightly larger than the diameter of the distal ring 110. According to the exemplary embodiment shown in FIG. 6, a diameter of the central portion 182 of the trough 130 is slightly smaller than the diameter of the distal ring 110, no diameter of the central portion 182 of the trough 130 is greater than the diameter of the distal ring 110, and a diameter of the terminal portion 180 of the trough 130 is slightly larger than the diameter of the central portion 182 of the trough, but slightly smaller than the diameter of the distal ring 110. According to the exemplary embodiment shown in FIG. 12, a diameter of the central portion 182 of the trough 130 is slightly smaller than the diameter of the distal ring 110, no diameter of the central portion 182 of the trough 130 is greater than the diameter of the distal ring 110, and a diameter of the terminal portion 180 of the trough 130 is slightly larger than the diameter of the distal ring 110. In some embodiments, a minimum diameter of the trough 130 is approximately 13-21 mm, or approximately 15-19 mm, or approximately 17-18 mm. In some embodiments, a maximum diameter of the trough 130 is approximately 13-21 mm, or approximately 15-19 mm, or approximately 17-18 mm.

Referring now to FIGS. 7A-7B and 13A-13B, a cross-section of the extraction device 100 (FIGS. 7A and 13A) and a cut-out cross-section of the distal ring 110 (FIGS. 7B and 13B) are shown. The sidewall 118 of the distal ring 110 is shown to include a taper such that the sidewall 118 decreases in thickness from proximate the distal opening 112 to the opposing opening 114 of the distal ring 110 (e.g., where the distal ring 110 transitions into the trough 130). In some embodiments, the sidewall 118 of the distal ring 110 includes a taper such that the thickness of the sidewall 118 proximate the distal opening 112 is approximately 3-5 mm or approximately 4 mm and the thickness of the sidewall 118 proximate the opposing opening 114 is approximately 2-3 mm or approximately 2.8 mm. The decrease in the width of the sidewall 118 from the distal opening 112 results in an increase in diameter of the hollow bore 116. By increasing the diameter of the hollow bore 116 from the distal opening 112 towards the trough 130, the distal ring 110 is better suited to receive the frame 14 and the prongs 12 of the stent graft 10. For example, because the hollow bore 116 increases in diameter as the frame 14 is received by the distal ring 110, the frame 14 contacts the inner surface 122 of sidewall 118 less and has less drag or friction with the sidewall 118. This allows the stent graft 10 to more easily slide (due to less friction or reduced drag) into the distal ring 110 and better prevents unexpected movement of the stent graft 10. In some embodiments, while the sidewall 118 decreases in thickness from the distal opening 112 towards the trough 130, the outside diameter of the sidewall 118 stays the same. As a result, the outer surface 120 of the sidewall 118 in contact with the walls of the aorta does not change in diameter for smoother manipulation.

Additionally, the distal ring 110 directly proximate the distal opening 112 includes a first blunt edge 124 (e.g., smooth edge, rounded edge, etc.). In use, the first blunt edge 124 is the first thing to come into contact with the stent graft 10 implanted in the vessel. As a result, it is important that the first blunt edge 124 is blunt or rounded to prevent a sharp contrast that may catch or become entangled with the frame 14, the vessel wall, and/or the prongs 12. In some embodiments, the first blunt edge 124 includes a radius of curvature of approximately 1-3 mm or approximately 2 mm. In this way, the first blunt edge 124 naturally comes into contact with the prongs 12 and moves them from the deployed position to the non-deployed position without catching or becoming entangled with the prongs 12. Similarly, the distal ring 110 directly proximate the opposing opening 114 may further include a second blunt edge 126 (for similar reasons as the first blunt edge 124). In this way, the prongs 12 do not catch on the first blunt edge 124 as it is being pulled through and out of the hollow bore 116 into the trough 130, which could pull on the entire extraction device 100.

In some embodiments, the extraction device 100 may contain an embossed (e.g., raised, extruded, etc.) or debossed (e.g., sunken, etc.) signifier. The signifier may be a brand name (e.g., logo, etc.) or an identifier of the extraction device 100 (e.g., the diameter of the distal ring, the length of L, etc.).

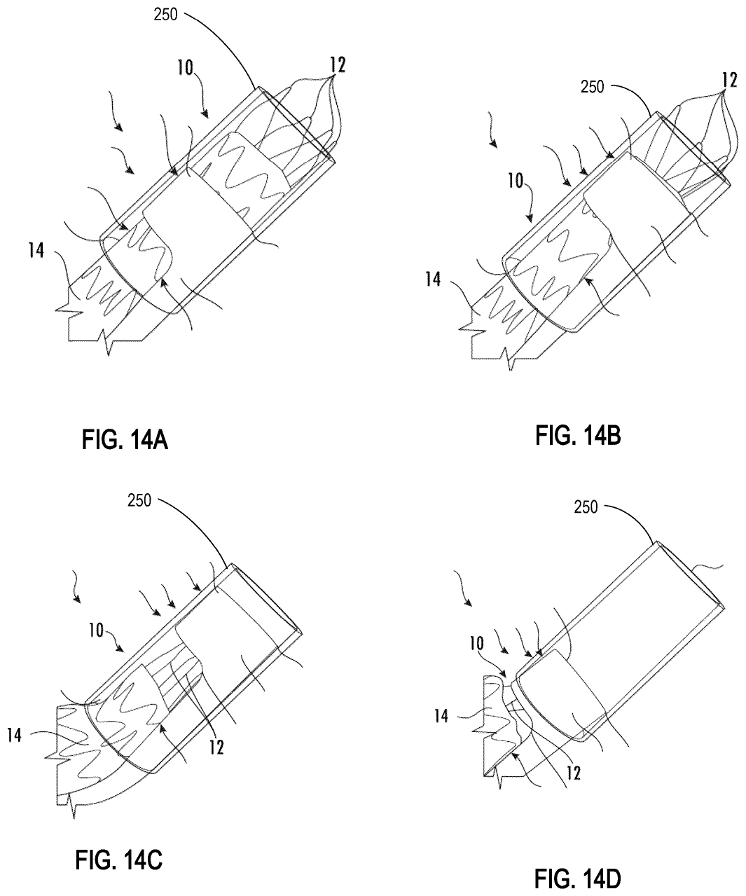

Referring now to FIGS. 14A-14D a method of extracting an endovascular stent graft (e.g., the stent graft 10) from a vessel 250 (e.g., one or more sections of the aorta, one or more arteries, etc.) is shown, according to an exemplary embodiment. First, a clamp (e.g., a forceps) is used to grasp a portion of the stent graft 10 (not shown), so as to hold the stent graft 10 in place during movement of the extraction device 100 over the stent graft 10, and to later (after the prongs 12 are moved to the non-deployed state) pull out the stent graft 10. In some embodiments, prior to this step, the vessel 250 itself is secured or clamped to prevent movement of the vessel 250 during the extraction. In other embodiments, multiple clamps are used to grasp a portion of the stent graft 10. In further embodiments, a clamp is used to grasp the extraction device 100.

Once the stent graft 10 is grasped by the clamp, an extraction device (e.g., the extraction device 100) is inserted into the vessel 250. The extraction device 100 is inserted into the vessel 250 through one or more incisions in the vessel 250 and is configured to fit inside of the vessel 250. In other embodiments and prior to use, the user of the extraction device 100 may determine the diameter of the vessel 250 and then determine which size of the extraction device 100 to use for insertion into the vessel 250.

Referring to FIGS. 14A and 14B, once the extraction device 100 has been inserted into the vessel 250, the extraction device 100 is slid over the stent graft 10 such that the distal ring 110 slides over the stent graft 10 and that the sidewall 118 is located between the stent graft 10 and a wall of the vessel 250 to compress the stent graft 10 within the distal ring 110. Once the distal opening 112 comes into contact with the frame 14 of the stent graft 10, the first blunt edge 124 and the taper of the sidewall 118 allow the frame 14 to easily slide (e.g., with little to no resistance) into the hollow bore 116.

Referring to FIG. 14C, after the extraction device 100 has been slid so the sidewall 118 is located between the stent graft 10 and the wall of the vessel 250, the extraction device 100 is further slid over the stent graft 10 until the extraction device 100 (e.g., first blunt edge 124 of the sidewall 118) causes a prong (e.g., the prongs 12) to release (e.g., move from the deployed position to the non-deployed position) from the wall of the vessel 250. To do so, the first blunt edge 124 of the sidewall 118 may push the prongs 12 radially inward and into the hollow bore 116 such that the sidewall 118 is between the prongs 12 and the wall of the vessel 250. In order to atraumatically remove the prongs 12, it is important that the prongs 12 do not catch on the sidewall 118 and therefore the sidewall 118 includes the first blunt edge 124 and also tapers from the distal opening 112 towards the trough 130. In other embodiments, other components of the extraction device 100 may be configured to remove the prongs 12 from the wall of the vessel 250.

Referring to FIG. 14D, once the prongs 12 are released from the wall of the vessel 250, the stent graft 10 and the extraction device 100 are fully removed from the vessel 250, concurrently or in sequence, with the sidewall 118 of the extraction device 100 located at least partially between the stent graft 10 and the vessel 250. The clamp may be used to grasp at least one of the stent graft 10 and/or the extraction device 100 for removal (e.g., the clamp is used to extract or pull out either one). In some embodiments, the stent graft 10 and the extraction device 100 are removed together, with the extraction device 100 continuing to push the prongs 12 radially inward to keep the prongs 12 from engaging the vessel 250 as it is slid out of the vessel 250. In this position, both the stent graft 10 and the extraction device 100 may be removed from the vessel 250. In such embodiments, the stent graft 10 may be extracted while within the hollow bore 116. In other embodiments, the stent graft 10 may be removed first, such that it is extracted from (e.g., is at least partially located within) the hollow bore 116 and through the trough 130. The stent graft 10 and the extraction device 100 are removed atraumatically from the vessel 250 such that they cause little to no damage to the vessel 250 itself. Because the sidewall 118 is located between the stent graft 10 and the vessel 250 as both are removed from the vessel 250, the vessel 250 does not (or minimally) comes into contact with the stent graft 10 and is better protected from damage. As compared to the stent graft 10 which includes the prongs 12, the sidewall 118 of the extraction device 100 is relatively smooth and therefore does not pull on or attach to the walls of the vessel 250.

Notwithstanding the embodiments described above with respect to the figures, various modifications and inclusions to those embodiments are contemplated and considered within the scope of the present disclosure.

It is also to be understood that the construction and arrangement of the elements of the systems and methods as shown in the representative embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed.

Accordingly, all such modifications are intended to be included within the scope of the present disclosure. Any means-plus-function clause is intended to cover the structures described herein as performing the recited function and not only structural equivalents but also equivalent structures. Other substitutions, modifications, changes, and omissions may be made in the design, operating conditions, and arrangement of the preferred and other illustrative embodiments without departing from scope of the present disclosure or from the scope of the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Similarly, unless otherwise specified, the phrase “based on” should not be construed in a limiting manner and thus should be understood as “based at least in part on.” Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

Moreover, although the figures show a specific order of method operations, the order of the operations may differ from what is depicted. Also, two or more operations may be performed concurrently or with partial concurrence. All such variations are within the scope of the disclosure.

What is claimed is:

1. A device for extracting an implant from a patient's circulatory system, comprising:
a cylindrical body having a distal opening, an opposing opening opposite the distal opening, and a sidewall surrounding a hollow bore of the cylindrical body;
a trough extending from the cylindrical body proximate the opposing opening having a first side wall and a second side wall; and
a handle extending from the trough;
wherein a thickness of the sidewall at the distal opening tapers toward the opposing opening such that the hollow bore proximate the distal opening is narrower than the hollow bore proximate the opposing opening such that a diameter of the opposing opening is larger than a diameter of the distal opening.

2. The device of claim 1, wherein the device follows a first curvature at the trough and a second curvature at the handle.

3. The device of claim 2, wherein the first curvature is a concave curvature relative to the trough and the second curvature is a convex curvature relative to the trough.

4. The device of claim 1, wherein the handle follows a convex curvature relative to the trough such that the handle curves away from the distal opening of the cylindrical body.

5. The device of claim 1, wherein the cylindrical body is approximately 14-18 millimeters in length.

6. The device of claim 1, wherein the cylindrical body is approximately 48-52 millimeters in length.

7. The device of claim 1, wherein the cylindrical body has an outer diameter of approximately 19-33 millimeters.

8. The device of claim 1, wherein a first blunt edge of the cylindrical body at the distal opening and a second blunt edge of the cylindrical body at the opposing opening are rounded to provide a smooth edge.

9. The device of claim 1, wherein the first side wall and the second side wall taper toward the handle such that a depth of the trough is greater proximate the cylindrical body than proximate the handle.

10. A method of extracting an implant from a vessel, comprising:
inserting an extraction device into the vessel, the extraction device comprising:
a cylindrical body having a distal opening, an opposing opening opposite the distal opening, and a sidewall surrounding a hollow bore of the cylindrical body;
a trough extending from the cylindrical body proximate the opposing opening having a first side wall and a second side wall; and
a handle extending from the trough;
wherein a thickness of the sidewall proximate the distal opening tapers toward the opposing opening such that the hollow bore proximate the distal opening is narrower than the hollow bore proximate the opposing opening such that the opposing opening has a diameter that is larger than a diameter of the distal opening;
sliding the extraction device over the implant until the extraction device causes the implant to release from the vessel; and
removing at least one of the implant or the extraction device from the vessel while the sidewall of the extraction device is located at least partially between the implant and the vessel.

11. The method of claim 10, wherein the implant comprises a replacement valve.

12. The method of claim 11, wherein the implant comprises an arterial stent graft.

13. The method of claim 12, wherein the first side wall and the second side wall taper toward the handle such that a depth of the trough is greater proximate the cylindrical body than proximate the handle.

14. The method of claim 10, wherein removing at least one of the implant and the extraction device from the vessel while the sidewall of the extraction device is located at least partially between the implant and the vessel further comprises removing the implant and the extraction device from the vessel concurrently.

15. The method of claim 10, further comprising 3-D printing the extraction device according to preferred specifications for a specific patient prior to inserting the extraction device into the vessel.

16. The method of claim 10, further comprising sterilizing the extraction device at least one of prior to inserting the extraction device into the vessel or after removing the extraction device from the vessel.

17. The method of claim 10, wherein the extraction device follows a first curvature at the trough and a second curvature at the handle.

18. The method of claim 17, wherein the first curvature is a concave curvature relative to the trough and the second curvature is a convex curvature relative to the trough.

19. The method of claim 12, wherein a first blunt edge of the cylindrical body at the distal opening and a second blunt edge of the cylindrical body at the opposing opening are rounded to provide a smooth edge.

20. A device for extracting an endovascular implant from a vessel, comprising:
a cylindrical body having a distal opening, an opposing opening opposite the distal opening, and a sidewall surrounding a hollow bore of the cylindrical body; and
a trough extending from the cylindrical body proximate the opposing opening having a first side wall and a second side wall;
wherein a thickness of the sidewall at the distal opening tapers toward the opposing opening such that the hollow bore proximate the distal opening is narrower than the hollow bore proximate the opposing opening thereby forming the opening opposite the distal opening with a diameter that is larger than a diameter of the distal opening; and
wherein the first side wall and the second side wall taper away from the cylindrical body such that a depth of the trough is greater proximate the cylindrical body than away from the cylindrical body.
